(19) United States
(12) Patent Application Publication
(10) Pub. No.: US 2013/0068219 A1
(43) Pub. Date: Mar. 21, 2013

(54) BYPASS HEAT AND MOISTURE EXCHANGER

(71) Applicants: Louis Javier Collazo, Pompano Beach, FL (US); Angelo R. Caruso, Boca Raton, FL (US)

(72) Inventors: Louis Javier Collazo, Pompano Beach, FL (US); Angelo R. Caruso, Boca Raton, FL (US)

(21) Appl. No.: 13/620,494

(22) Filed: Sep. 14, 2012

Related U.S. Application Data
(60) Provisional application No. 61/534,840, filed on Sep. 14, 2011.

(51) Int. Cl. A61M 16/10; A61M 16/20; A61M 16/00

(52) U.S. Cl. CPC A61M 16/1045; A61M 16/0057; A61M 16/20; A61M 2016/201
USPC 128/201.13

(57) ABSTRACT

A heat and moisture exchanger (HME) device for respiratory therapy, the device comprising a housing defining a passageway for the flow of respiratory gas, where the housing can include a ventilator side port, a patient side port, a first pathway defined within the housing, and a second pathway defined within the housing, a medium located within the first pathway and a valve configured to selectably impede fluid flow through one of the first pathway and second pathway during use. The HME can further include a knob configured to position the valve. The housing can include a first sealing wall and a second sealing wall, where the valve is configured to abut the first sealing wall in a first valve position and to abut the second sealing wall in a second valve position.

### CROSS-REFERENCE TO RELATED APPLICATIONS

This application claims the benefit of U.S. Provisional Patent Application Ser. No. 61/534,840, filed on Sep. 14, 2011, the entirety of which is incorporated by reference herein.

### BACKGROUND OF THE INVENTION

1. Statement of the Technical Field

The present invention relates generally to systems for respiratory therapy, and more particularly to a bypass heat and moisture exchanger device for providing humidification and aerosol medication to a ventilator system.

2. Description of the Related Art

In the current practice of ventilator care, a HME (heat and moisture exchanger) is a common way to humidify the patient's respiratory gasses. An HME allows gas to flow through in both directions, but not humidity (water vapor). The HME uses a filter media (e.g., a porous material) to trap moisture in the exhaled breath of a patient and recycle the humidity for the next breath of that patient. For treating some respiratory conditions, it is desirable to deliver aerosol medication to the ventilated patient by attaching a nebulizer or other aerosol delivery apparatus to a ventilator circuit.

Currently, the clinician must disconnect the HME from the circuit before delivering the aerosol medication because the filter media in the HME does not allow for aerosol to effectively pass through. In addition, if aerosol is delivered with the HME in place, the HME media would become saturated with moisture and the flow resistance in the ventilator circuit will drastically increase.

The process of opening the ventilator circuit to atmosphere in order to replace or remove components is recognized as potentially harmful to the patient for various reasons, such as lung derecruitment, hypoxemia, and other potentially harmful effects. Clinicians strive to keep the circuit intact for as long as possible when caring for the ventilated patient. Therefore, it is highly advantageous to be able to deliver aerosol medication without disconnecting the ventilator circuit.

### SUMMARY OF THE INVENTION

Embodiments of the present invention address deficiencies of the art in respect to ventilator systems, and provide a novel and non-obvious apparatus, system, and method for providing heat and moisture exchanger (HME) and aerosol medications in airways of ventilated patients. In an embodiment of the invention, a bypass heat and moisture exchanger device for connecting to a ventilator can be provided. The bypass heat and moisture exchanger device can include a housing defining a passageway for the flow of respiratory gas, where the housing includes a ventilator side port, a patient side port, a first pathway defined within the housing, and a second pathway defined within the housing, a medium located within the first pathway and a valve configured to selectably impede fluid flow through one of the first pathway and second pathway during use. The HME can further include a knob configured to position the valve. The housing can include a first sealing wall and a second sealing wall, where the valve is configured to abut the first sealing wall in a first valve position and to abut the second sealing wall in a second valve position.

In another embodiment, the bypass heat and moisture exchanger device can include a housing having an upper housing portion and a lower housing portion coupled to the lower housing portion, the lower housing portion can include a first side port and a second side port opposite the first side port. The housing can define a first lumen which defines a first gas pathway and a second lumen which defines a second gas pathway. The first lumen can be defined by the outer wall of the housing and a first internal wall. Similarly the second lumen can be defined by the outer wall of the housing and a second internal wall spaced apart from the first internal wall. The space between the first internal wall and the second wall can vary in distance. The bypass heat and moisture exchanger device further can include a valve assembly that rotates from a first sealing position to a second sealing position about a hinge portion.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is a perspective view of a bypass heat and moisture exchanger (HME) device constructed in accordance with an embodiment of the present invention;

FIG. 2 is an exploded view of the bypass heat and moisture exchanger (HME) device of FIG. 1 illustrating the major components in disassembled relationship;

FIG. 3A is a top view of the bypass heat and moisture exchanger (HME) device of FIG. 1, which is in the HME mode;

FIG. 3B is a top view of the bypass heat and moisture exchanger (HME) device of FIG. 1, which is in the aerosol mode;

FIG. 4A is a side view of the bypass heat and moisture exchanger (HME) device of FIG. 1, which is constructed in accordance with an embodiment of the present invention;

FIG. 4B is an end view of the bypass heat and moisture exchanger (HME) device of FIG. 1;

FIG. 5A is a perspective sectional view of the bypass heat and moisture exchanger (HME) device of FIG. 1, which illustrates a valve closing the aerosol pathway;

FIG. 5B is a perspective sectional view of the bypass heat and moisture exchanger (HME) device of FIG. 1, which illustrates a valve closing the HME pathway;

FIG. 6A is a sectional end view of the bypass heat and moisture exchanger (HME) device of FIG. 1; and

FIG. 6B is a longitudinal sectional side view of the bypass heat and moisture exchanger (HME) device of FIG. 1.

### DETAILED DESCRIPTION OF THE INVENTION

Embodiments of the present invention address deficiencies of the art in respect to ventilator systems, and provide a novel and non-obvious apparatus, system, and method for providing heat and moisture exchanger (HME) and aerosol medications in airways of ventilated patients. In an embodiment of the invention, a bypass heat and moisture exchanger device for connecting to a ventilator can be provided. The bypass heat and moisture exchanger device can include a housing defining a passageway for the flow of respiratory gas, where the housing includes a ventilator side port, a patient side port, a first pathway defined within the housing, and a second pathway defined within the housing, a medium located within the first pathway and a valve configured to selectably impede fluid flow through one of the first pathway and second pathway during use. The HME can further include a knob configured to position the valve. The housing can include a first sealing wall and a second sealing wall, where the valve is configured to abut the first sealing wall in a first valve position and to abut the second sealing wall in a second valve position.

The bypass heat and moisture exchanger device can include a housing having an upper housing portion and a lower housing portion coupled to the lower housing portion, the lower housing portion can include a first side port and a second side port opposite the first side port. The housing can define a first lumen which defines a first gas pathway and a second lumen which defines a second gas pathway. The first lumen can be defined by the outer wall of the housing and a first internal wall. Similarly the second lumen can be defined by the outer wall of the housing and a second internal wall spaced apart from the first internal wall. The space between the first internal wall and the second wall can vary in distance. The bypass heat and moisture exchanger device further can include a valve assembly that rotates from a first sealing position to a second sealing position about a hinge portion.

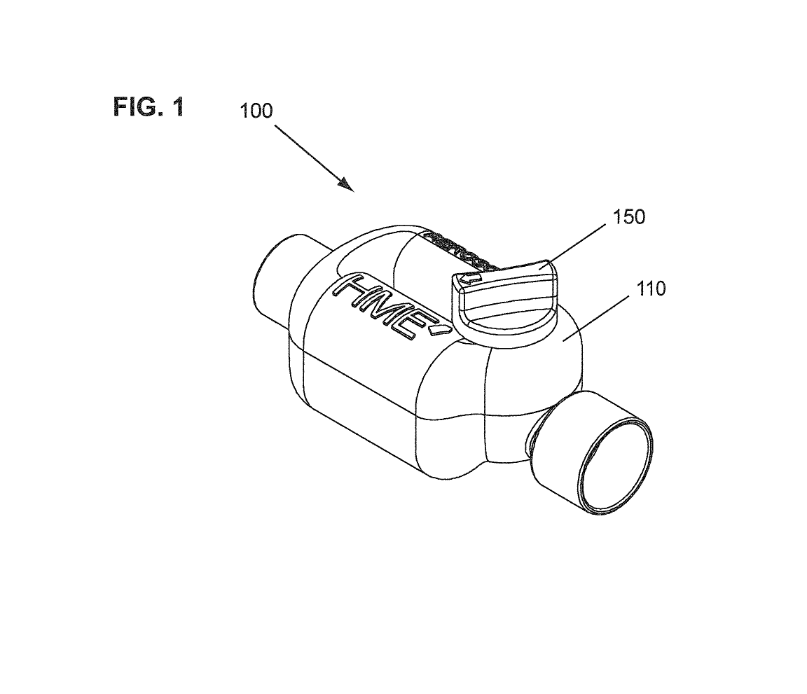

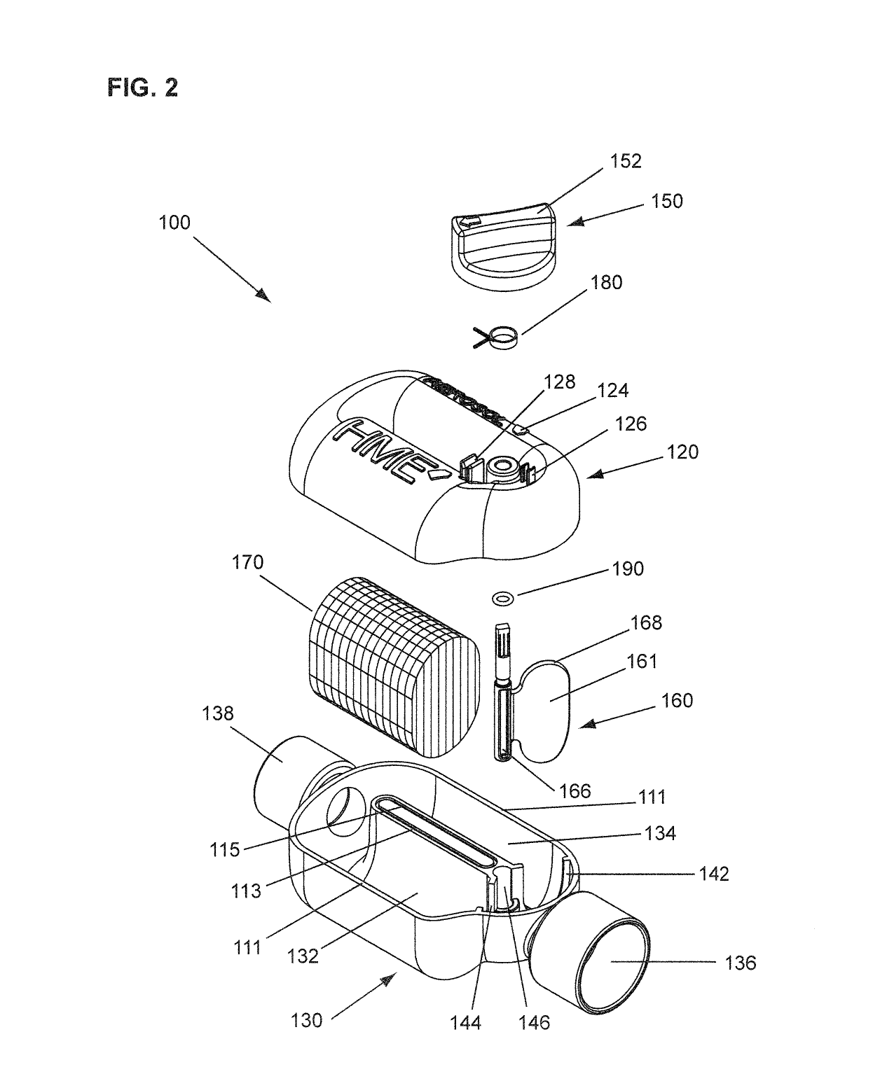

In illustration, FIG. 1 is a perspective view of a bypass heat and moisture exchanger (HME) device constructed in accordance with an embodiment of the present invention. The bypass heat and moisture exchanger (HME) 100 can include a housing 110 and a knob 150. FIG. 2 is an exploded view of the bypass heat and moisture exchanger (HME) device of FIG. 1 illustrating the major components in disassembled relationship. As illustrated by FIG. 2, housing 110 can include an upper housing portion 120 and a lower housing portion 130. As illustrated in FIG. 2, upper housing portion 120 and lower housing portion 130 are separate components that are assembled to produce a complete housing 110. For example, upper housing portion 120 and lower housing portion 130 can be welded to form housing 110. In embodiments, housing 110 also can be a single part, for example a one piece blow-molded housing. Various materials can be used to manufacture the housing 110 including clear or transparent material. The housing further can be rigid or flexible. Housing 110 defines two lumens or pathways 132 and 134 in which gas can flow. Pathway 132 is defined as a HME pathway which includes a HME filter 170. Pathway 134 is defined as an aerosol pathway, which is an alternate pathway to HME pathway 132, and is utilized when the user wants to bypass HME filter 170. The first lumen or pathway 132 is defined within housing 110 by outside wall 111 and first internal wall 113. Similarly, the second lumen or pathway 134 is defined within housing 110 by outside wall 111 and second internal wall 115. In embodiments, first internal wall 113 and second internal wall 115 can be the same wall. Advantageously, the gas pathways 132 and 134 are designed such that the size of one pathway will not impact the size or function of the other pathways. In this sense the pathways are structured as independent lumens in one housing 110. The size (i.e., diameter) of one lumen can be changed without impacting the size of the other lumen. In addition, the pathways 132 and 134 do not need to share a common axis or have axes that are on the same plane in one or more directions. Also, the axes do not have to be parallel. The lower housing portion 130 can have a first side port 136, e.g., a ventilator side port and a second side port 138, e.g., a patient side port.

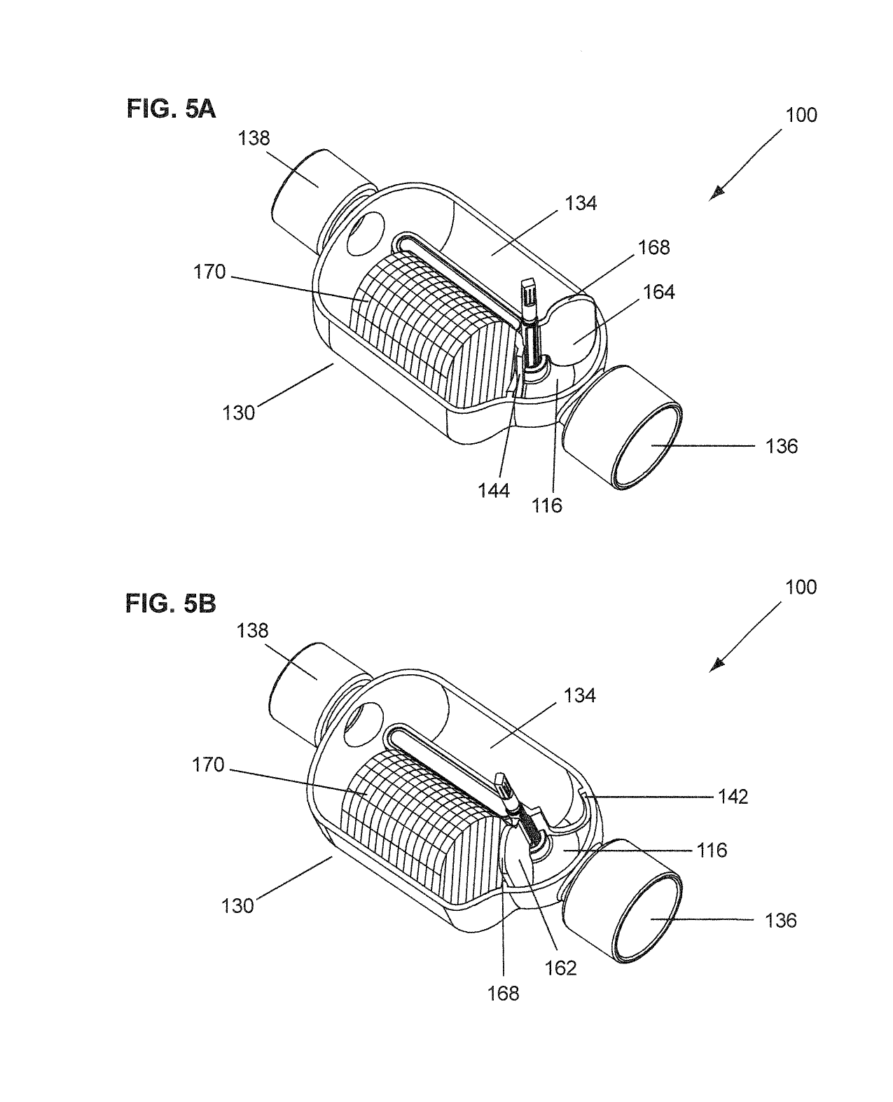

Housing 110 can include a first sealing wall 142 and a second sealing wall 144. Both first sealing wall 142 and second sealing wall 144 extend from the inside surface of outside wall 111 and form ring-like portions similar to a door hatch to a ship or submarine. The angle between first sealing wall 142 and second sealing wall 144 is less than 180 degrees which lessens the amount that the user has to actuate knob 150 and therefore facilitates switching between HME mode and aerosol mode. It is preferred that this angle be greater than 90 degrees, for example 120 degrees. In embodiments, neither first sealing wall 142 nor second sealing wall 144 is normal and/or parallel to the axis of first side port 136. Housing 110 further can include a valve receptacle 146 which is configured to receive a valve assembly 160. Various materials can be used to manufacture the valve assembly 160 including clear or transparent material. It is preferred that valve assembly be tinted so it can be more easily seen by the user through housing 110. The valve assembly 160 further can be rigid or flexible. As illustrated in FIG. 2, valve receptacle 146 can receive a hinge portion 166 of valve assembly 160. With the device in its assembled state, the hinge portion 166 protrudes from the housing 110 and couples to the knob 150 for example via a press-fit. The valve assembly 160 can include the hinge portion 166 attached to one edge of valve flap 161. It is preferred that the hinge portion 166 be adjacent to valve flap 161 so that hinge portion 166 is not in the gas paths where it could impede flow. The valve flap 161 can include a first sealing surface 162 (best shown in FIG. 5B) and a second sealing surface 164 (best shown in FIG. 5A) opposite the first sealing surface 162. The user can rotate the valve assembly 160 (by rotating the knob 150) so that the first sealing surface 162 can stop and seal against housing first sealing wall 142. This blocks or impedes the aerosol pathway 134 as shown in FIG. 5A and forces the gas to flow through the HME pathway 132. The user can also rotate the valve assembly 160 so the second sealing surface 164 can stop and seal against housing second sealing wall 144. This blocks or impedes the HME pathway 132 as shown in FIG. 5B and forces the gas to flow through the aerosol pathway 134. In this matter, the valve assembly selectively impedes fluid flow through one of the first pathway and the second pathway. In either position shown in FIG. 5A and 5B, an end surface 168 of valve flap 161 is maintained out of the desired gas path. In embodiments, end surface 168 can seal against an inner surface 116. This sealing could be in conjunction with and/or instead of the sealing that occurs at the sealing walls 142 and 144. Upper housing portion 120 can include a lock feature 126 and a stop wall 128 on the outside surface of outside wall 111. Bypass HME device 100 can further include knob 150 that resides on the outside surface of outside wall 111 of housing 110 and is coupled to a top section of hinge portion 166 of valve assembly 160. In embodiments, a second valve assembly (not shown) which would be similar to the first valve assembly 160 can seal the opposite side of the same pathway which is selected by the user for sealing. In this embodiment, it would be advantageous to have both valves actuated by rotating knob 150.

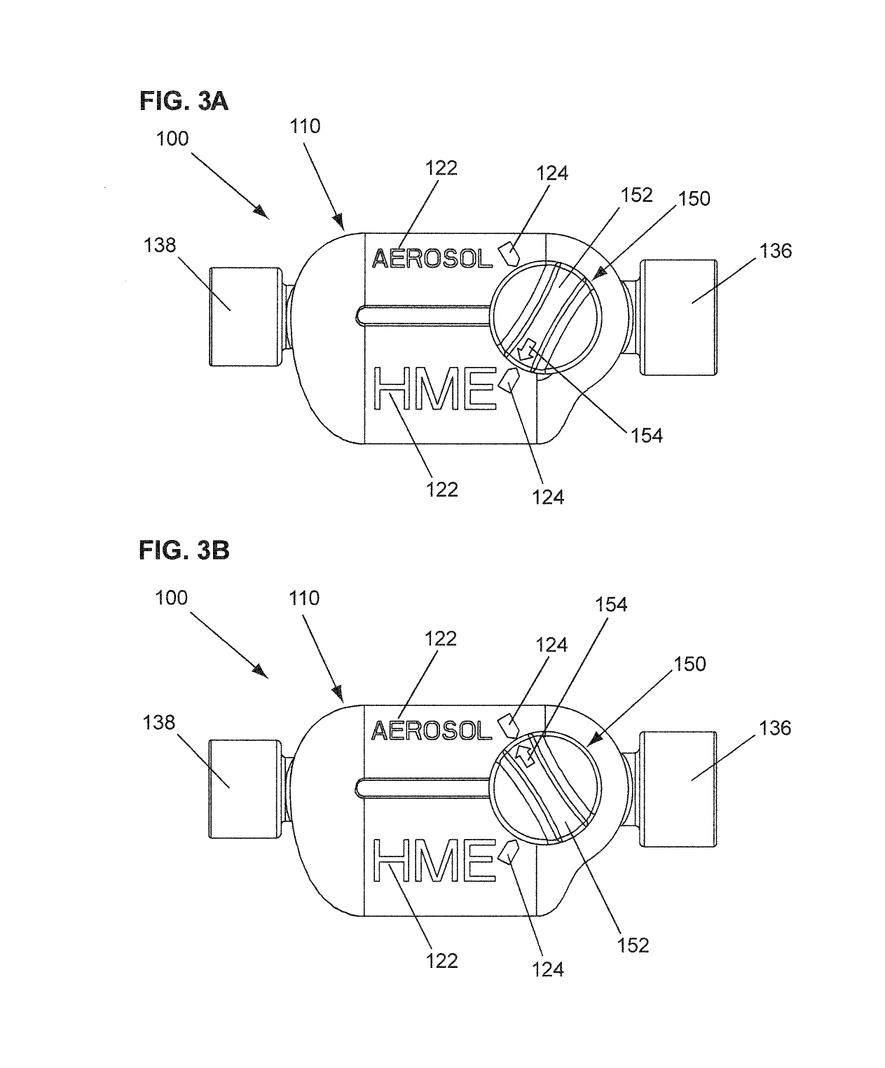

The stop wall 128 on housing 110 can limit the range of rotational travel of knob 150 in one or more directions. The lock feature 126 on housing 110 can maintain knob 150 (and valve assembly 160) in a desired rotational position. Knob 150 can be manufactured from various materials, including rigid and flexible materials. The knob 150 can actuate the valve assembly 160 and indicate the mode in which the bypass HME device 100 currently resides. As illustrated in FIGS. 3A and 3B, knob 150 can include a selection arrow 154, which can be used in conjunction with pathway indicators 124 to indicate which mode the bypass HME device 100 currently is operating. For example, FIG. 3A illustrates the bypass HME device 100 in HME mode while FIG. 3B illustrates the bypass HME device 100 in aerosol mode. Bypass HME device 100 can further include markings 122 that reside on the outside surface of outside wall 111 of housing 110 and that help an user to identify which pathway is the HME pathway 132 and which pathway is the aerosol pathway 134. In embodiments, bypass HME device 100 can include spring 180, as well as an o-ring 190 that seals the hinge portion 166 to the housing 110. Spring 180 is coupled between knob 150 and hinge portion 166, where the spring 180 helps to maintain knob 150 (and valve assembly 160) in a desired rotational position. Spring 180 can be any type of spring, e.g., a torsional spring, and/or any known method that can maintain the knob under a load, e.g., a compressed rubber part. Spring 180 also ensures that valve flap 161 is maintained, after the user releases the knob 150, in contact with one of the first sealing wall 142 and the second sealing wall 144. This prevents a position such as one where the valve flap 161 is maintained adjacent to the first side port 136. Knob 150 can have a lock feature (not shown) that mates with lock feature 126 on housing 110 to maintain knob 150 (and valve assembly 160) in a desired rotational position. Notably, knob 150, spring 180 and hinge portion 166 are not in the gas pathways 132 and 134. Therefore, no portion of the actuation device is in the gas paths.

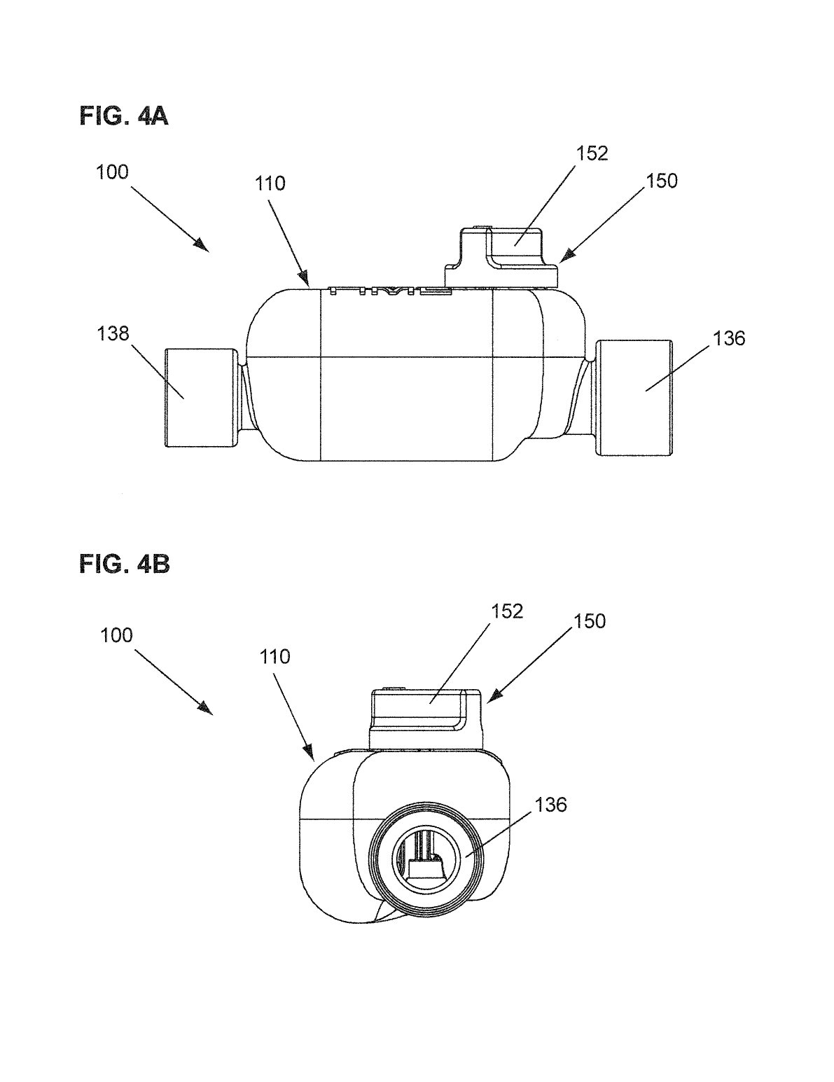

FIG. 4A is a side view of the bypass heat and moisture exchanger (HME) device of FIG. 1. While FIG. 4B is an end view of the bypass heat and moisture exchanger (HME) device of FIG. 1. FIGS. 5A and 5B are perspective sectional views of the bypass heat and moisture exchanger (HME) device of FIG. 1 where the upper portion 120 and knob 150 are not included for clarity. FIG. 5A illustrates a valve flap 161 closing the aerosol pathway 134. FIG. 5B illustrates a valve flap 161 closing the HME pathway. When one pathway is closed, the airflow is directed through the other pathway.

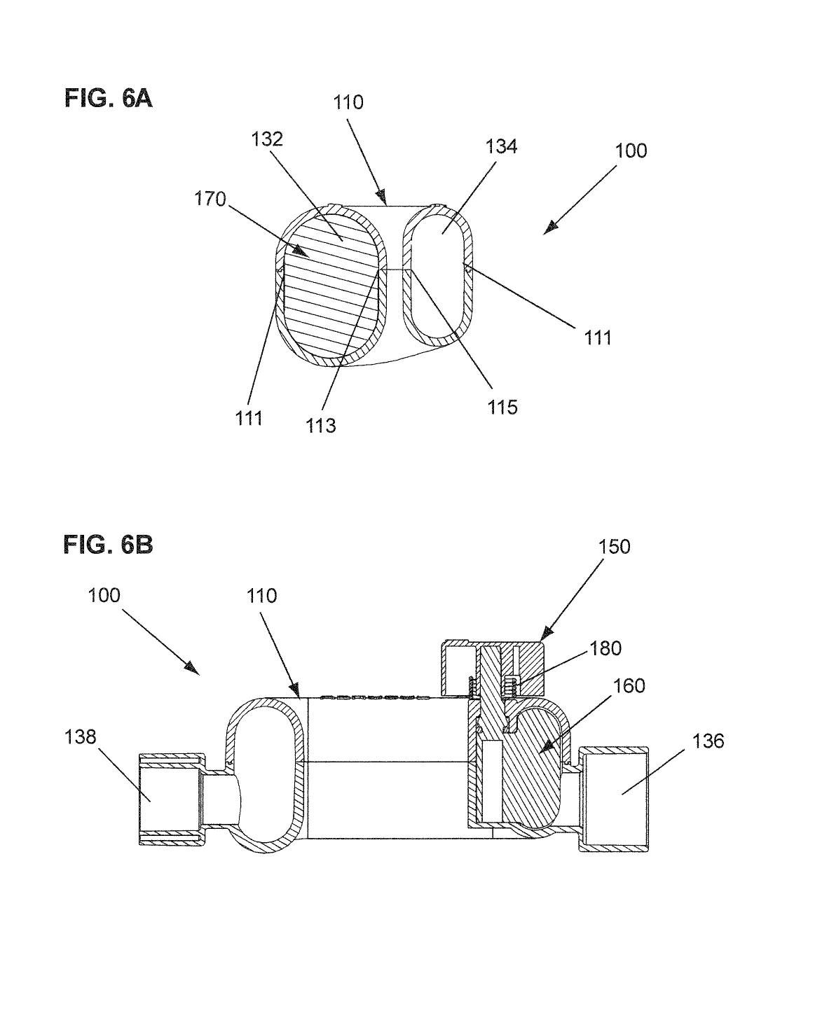

FIG. 6A is a cross-sectional view of the HME device 100 through the HME and aerosol pathways. This figure illustrates that HME pathway 132 and aerosol pathway 134 are distinct pathways and can be different size and shape. HME pathway 132 and the aerosol pathway 134 need not be coaxial. In fact it is advantageous to separate the different pathways, so that the operator can more easily tell which pathway is selected, and which pathway the gas is flowing through. FIG. 6B is a cross-sectional view of the device through the hinge portion 166 of the valve assembly 160.

In operation, the Bypass HME 100 as described is a device that allows the clinician to deliver aerosol medications without opening the ventilator circuit to disconnect the HME. The Bypass HME device 100 connects to the ventilator circuit in the same manner and location as a standard HME. During normal operation, the gas path is routed through the HME filter 170 and operates to retain the humidity in the patient's breath as with a traditional HME. When the clinician wants to deliver aerosol, they actuate a valve that puts the Bypass HME device into an “aerosol” mode. In this mode, the gas flow is routed through another pathway that does not contain the HME media; therefore the HME is “bypassed”. When the clinician is finished delivering the aerosol medication, the clinician simply actuates the valve to return the device to “HME” mode. The described device is advantageous over prior art because the valve does not impede flow (i.e., increase resistance) in the pathway through which gas is intended to flow. Also, it is advantageous because it completely isolates (as opposed to partially isolates) the HME from the inspiratory gas flow when set to aerosol mode. Because of its double non-coaxial pipe shape it also allows clear visualization of each gas flow pathway. The valve can also be seen through the housing, clearly indicating which pathway the respiratory gasses are flowing through. Knob 150 has a holding portion 152 which is intended to be parallel to valve flap 161. Because they are parallel, the direction of the holding portion 152 more clearly communicates to the user the direction of the valve and hence the mode of the device. In embodiments, a bacterial or viral filter (not shown) can be placed between the HME filter 170 and second sealing wall 144. Further, housing 110 is shaped to optimize holding and controlling in order to facilitate user connection to the ventilator circuit and actuation of the knob 150. In embodiments, features such as grooves and protrusions can exist in the housing 110 to further enhance holding and controlling.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims, which are to be accorded with the broadest interpretation so as to encompass all such modifications and similar structures.

We claim:

1. A heat and moisture exchanger device for respiratory therapy, the device comprising:
a housing defining a passageway for the flow of respiratory gas, wherein the housing includes:
a ventilator side port;
a patient side port;
a first pathway defined within the housing;
a second pathway defined within the housing;
a medium located within the first pathway; and
a valve configured to selectably impede fluid flow through one of the first pathway and second pathway during use.

2. The heat and moisture exchanger device of claim 1, wherein the valve is located outside of the one of the first pathway and second pathway.

3. The heat and moisture exchanger device of claim 1, wherein the valve is located outside of the first pathway and second pathway.

4. The heat and moisture exchanger device of claim 1, wherein the valve has at least two surfaces configured for sealing with other surfaces.

5. The heat and moisture exchanger device of claim 1, wherein the valve has a first surface configured for sealing the first pathway and a second surface configured for sealing the second pathway.

6. The heat and moisture exchanger device of claim 1, further comprising a knob configured to position the valve.

7. The heat and moisture exchanger device of claim 6, wherein the knob has a portion that indicates the position of a flap on the valve.

8. The heat and moisture exchanger device of claim 6, wherein the knob has a portion that aligns with an indicator on the housing to indicate the position of a flap on the valve.

9. The heat and moisture exchanger device of claim 1, wherein the first pathway and the second pathway have a different axis.

10. The heat and moisture exchanger device of claim 1, wherein the first pathway and the second pathway have a different axis.

11. The heat and moisture exchanger device of claim 1, wherein the housing has a first sealing wall and a second sealing wall, and wherein the valve is configured to abut the first sealing wall in a first valve position and to abut the second sealing wall in a second valve position.

12. The heat and moisture exchanger device of claim 1, wherein the housing has a first sealing wall and a second sealing wall, and wherein an angle between the first sealing wall and the second sealing wall is less than 180 degrees.

13. The heat and moisture exchanger device of claim 1, wherein the housing has a first sealing wall and a second sealing wall, and wherein an angle between the first sealing wall and the second sealing wall is in the range of 90 degrees to 180 degrees.

14. The heat and moisture exchanger device of claim 1, wherein the housing has a first sealing wall and a second sealing wall, and wherein the ventilator side port has a ventilator side port axis, wherein both the first sealing wall and the second sealing wall are other than one of normal and parallel to the ventilator side port axis.

15. The heat and moisture exchanger device of claim 1, wherein when the valve is positioned to impede flow through one of the pathways, the valve does not impede flow through the other pathway.

16. The heat and moisture exchanger device of claim 1, wherein further comprising a spring that is external of the housing.

17. The heat and moisture exchanger device of claim 1, wherein further comprising a spring that is outside of the flow of respiratory gas.

18. A heat and moisture exchanger device for respiratory therapy, the device comprising:
a housing defining a passageway for the flow of respiratory gas, wherein the housing includes:
a ventilator side port;
a patient side port;
a first pathway defined within the housing;
a second pathway defined within the housing;
a medium located within the first pathway; and
a valve configured for providing an user with control of the flow of the respiratory gas through the device.

18. A heat and moisture exchanger device for respiratory therapy, the device comprising:
a housing defining a passageway for the flow of respiratory gas, wherein the housing includes:
a ventilator side port;
a patient side port;
a first pathway defined within the housing;
a second pathway defined within the housing;
a medium located within the first pathway; and
a valve configured for providing an user to select between a first position to impede fluid flow through the first path and a second position to impede fluid flow through the second path.
